(19) United States
(12) Patent Application Publication
(10) Pub. No.: US 2017/0020394 A1
(43) Pub. Date: Jan. 26, 2017

(54) MECHANICALLY AND/OR MAGNETICALLY NAVIGABLE CATHETER WITH FIBER OPTIC POSITION OR SHAPE SENSORS

(71) Applicant: STEREOTAXIS, INC., St. Louis, MO (US)

(72) Inventor: Scott Harrington, St. Louis, MO (US)

(21) Appl. No.: 15/201,212

(22) Filed: Jul. 1, 2016

Related U.S. Application Data
(60) Provisional application No. 62/199,221, filed on Jul. 30, 2015.
(60) Provisional application No. 62/189,659, filed on Jul. 7, 2015.

(51) Int. Cl. A61B 5/00; A61N 1/05; A61B 5/042; A61B 5/06; A61M 25/01

(52) U.S. Cl. CPC A61B 5/0084; A61B 5/065; A61M 25/0147; A61M 25/0158; A61B 5/042; A61N 1/05

(57) ABSTRACT

A mechanically and/or magnetically navigable catheter has an elongate body having a proximal and a distal end. At least one mechanically or magnetically responsive element is associated with the distal end of the elongate body for navigating the distal end in the body. At least one fiber optic sensor extends substantially along the length of the elongate body for use determining the location of at least one point along the length of the elongate body, which can be used as an input an automated navigation system.

### CROSS-REFERENCE TO RELATED APPLICATION

This application claims priority to U.S. Provisional Patent Application No. 62/189,659, filed on Jul. 7, 2015 and U.S. Provisional Patent Application No. 62/199,221, filed on Jul. 30, 2015. The entire disclosures of the above applications are incorporated herein by reference.

### FIELD OF THE INVENTION

The present disclosure relates to mechanically and/or magnetically navigable catheter with fiber optic position or shape sensors, and in particular to mechanically or magnetically navigable catheters incorporating fiber optic shape position detecting and/or shape sensing.

### BACKGROUND

This section provides background information related to the present disclosure which is not necessarily prior art.

Mechanically navigable catheters are elongate medical devices that incorporate one or more mechanically actuated elements, typically operated by one or more push wires or pull wires accessible at the proximal end, to bend or twist the device to orient or position the distal end. Magnetically navigable catheters are elongate medical devices that incorporate one or more magnetically responsive elements that allow the catheter to bend or twist in response to an applied magnetic field or gradient, so that the distal end of the device can be oriented or positioned. The magnetically responsive elements can be one or more discrete magnets incorporated at the distal end, or along the distal end portion of the catheter. Alternatively the magnetically responsive elements can be magnetically responsive material incorporated into portions of the catheters. Still another alternative for the magnetically responsive elements are small electromagnets-incorporated at the distal end, or along the distal end portion of the catheter.

Fiber optic position and shape sensors have been developed, some of which allow the location of a particular point along the fiber to be determined, and others of which allow the location of all the points along the fiber to be determined, thereby defining the shape of the fiber in three dimensional space. On such sensor uses low reflectance Fiber Bragg Grating (FBG) strain sensors in a multi-core fiber to determine how any point along that fiber is positioned in space. The characteristics of optical fibers and the FBGs vary with curvature, and by sensing the relative change of FBGs in each of one or more fiber cores, the three-dimensional change in position can be determined. By using this method, precise deflection, end position, and location can be determined in real time even in fibers that may be experiencing external twisting.

### SUMMARY

This section provides a general summary of the disclosure, and is not a comprehensive disclosure of its full scope or all of its features.

Generally embodiments of this invention provide a mechanically or magnetically navigable catheter with position or shape sensing capabilities. The catheter comprises an elongate body having a proximal and a distal end. A mechanically navigable catheter includes at least one mechanically operated element incorporated in the elongate body to change the shape or configuration body, and at least one elongate element, such as a push wire or a pull wire to operate the mechanically operated element. A magnetically navigable catheter includes at least one magnetically responsive element that is associated with the distal end of the elongate body. At least one fiber optic sensor extends substantially along the length of the elongate body for use determining the location of at least one point along the length of the elongate body.

There are preferably a plurality of fiber optic sensors, and more preferably three fiber optic sensors extending substantially along the length of the elongate body. In the preferred embodiment the fiber optic sensors extend longitudinally, parallel to the longitudinal axis of the elongate body.

In some embodiments the fiber optic sensors are spaced equally around the cross-sectional perimeter of the elongate body, although in other embodiments the fiber optic sensors are not equally spaced. For example in one preferred embodiment there are at least first, second and third fiber optic sensors, the second and third sensors being offset 90° on opposite sides of the first sensor. In other embodiments, the at least two of the fiber optic sensors are disposed adjacent to each other, and more preferably three fiber optic sensors are disposed adjacent to each other.

In some embodiments the catheter comprises a cladding surrounding the elongate body, and the fiber optic sensors are embedded in this cladding. In other embodiments, the fiber optic sensors are secured on the outside of the elongate body with a sleeve enveloping the fiber optic sensors and the elongate body. This sleeve can comprise a polymeric web material. The sleeve can alternatively comprise a mesh material, made of metal, polymer, or some combination.

In still other embodiments, the elongate body can have at least one groove therein, at least partially receiving at least one fiber optic sensor. Preferably there are a plurality of grooves therein, each at least partially receiving at least one fiber optic sensor. In some embodiments the groove is sized sufficiently to receive substantially the entire sensor or multiple sensors.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a transverse cross sectional view of a first preferred embodiment of a magnetically navigable catheter with fiber optic position or shape sensor in accordance with the principles of this invention;

FIG. 2 is a transverse cross sectional view of a second preferred embodiment of a magnetically navigable catheter with fiber optic position or shape sensor in accordance with the principles of this invention;

FIG. 3 is a transverse cross sectional view of a third preferred embodiment of a magnetically navigable catheter with fiber optic position or shape sensor in accordance with the principles of this invention;

FIG. 4 is a transverse cross sectional view of a fourth preferred embodiment of a magnetically navigable catheter with fiber optic position or shape sensor in accordance with the principles of this invention;

FIG. 5 is a transverse cross sectional view of a fifth preferred embodiment of a magnetically navigable catheter with fiber optic position or shape sensor in accordance with the principles of this invention;

FIG. 6 is a transverse cross sectional view of a sixth preferred embodiment of a magnetically navigable catheter with fiber optic position or shape sensor in accordance with the principles of this invention;

FIG. 7 is a transverse cross sectional view of a seventh preferred embodiment of a magnetically navigable catheter with fiber optic position or shape sensor in accordance with the principles of this invention;

FIG. 8 is a transverse cross sectional view of a eighth preferred embodiment of a magnetically navigable catheter with fiber optic position or shape sensor in accordance with the principles of this invention;

FIG. 9 is a transverse cross sectional view of a ninth preferred embodiment of a magnetically navigable catheter with fiber optic position or shape sensor in accordance with the principles of this invention;

FIG. 10 is a longitudinal cross sectional view of a tenth preferred embodiment of a magnetically navigable catheter with fiber optic position or shape sensor in accordance with the principles of this invention;

FIG. 11 is a transverse cross sectional view of a first preferred embodiment of a mechanically navigable catheter with fiber optic position or shape sensor in accordance with the principles of this invention;

FIG. 12 is a transverse cross sectional view of a second preferred embodiment of a mechanically navigable catheter with fiber optic position or shape sensor in accordance with the principles of this invention;

FIG. 13 is a transverse cross sectional view of a third preferred embodiment of a mechanically navigable catheter with fiber optic position or shape sensor in accordance with the principles of this invention;

FIG. 14 is a transverse cross sectional view of a fourth preferred embodiment of a mechanically navigable catheter with fiber optic position or shape sensor in accordance with the principles of this invention;

FIG. 15 is a transverse cross sectional view of a fifth preferred embodiment of a mechanically navigable catheter with fiber optic position or shape sensor in accordance with the principles of this invention;

FIG. 16 is a transverse cross sectional view of a sixth preferred embodiment of a mechanically navigable catheter with fiber optic position or shape sensor in accordance with the principles of this invention;

FIG. 17 is a transverse cross sectional view of a seventh preferred embodiment of a mechanically navigable catheter with fiber optic position or shape sensor in accordance with the principles of this invention;

FIG. 18 is a transverse cross sectional view of a eighth preferred embodiment of a mechanically navigable catheter with fiber optic position or shape sensor in accordance with the principles of this invention;

FIG. 19 is a transverse cross sectional view of a ninth preferred embodiment of a mechanically navigable catheter with fiber optic position or shape sensor in accordance with the principles of this invention; and

FIG. 20 is a longitudinal cross sectional view of a tenth preferred embodiment of a mechanically navigable catheter with fiber optic position or shape sensor in accordance with the principles of this invention.

Corresponding reference numerals indicate corresponding parts throughout the several views of the drawings.

### DETAILED DESCRIPTION

Example embodiments will now be described more fully with reference to the accompanying drawings.

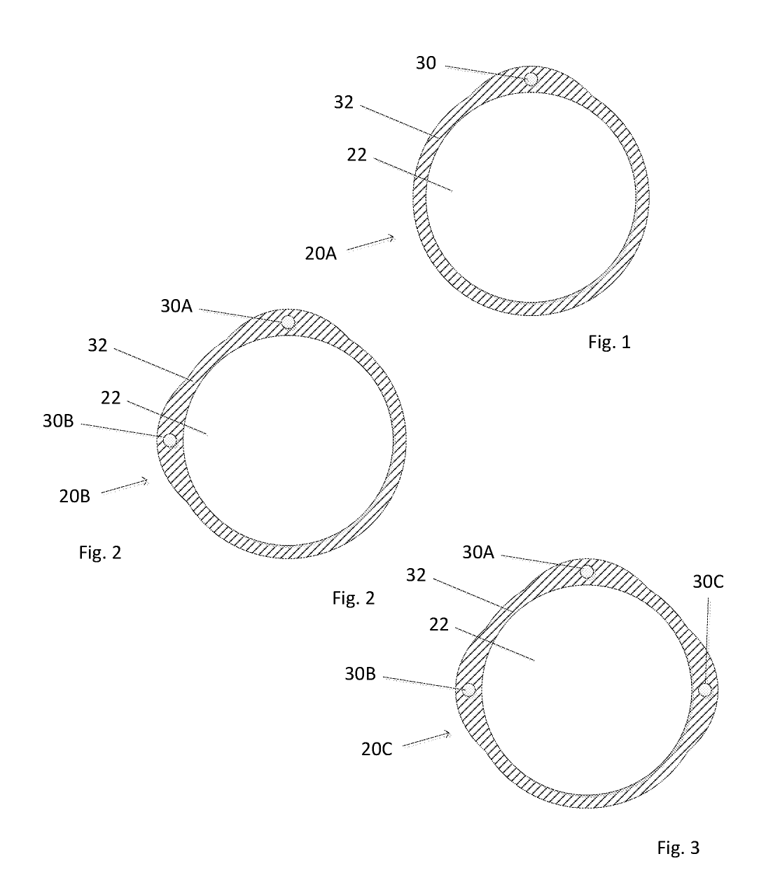

A first preferred embodiment of a magnetically navigable catheter with fiber optic position or shape sensor in accordance with the principles of this invention, indicated generally as 20A, is shown in transverse cross section in FIG. 1. The magnetically navigable catheter 20A comprises an elongate body 22 having a proximal end, and a distal end. At least one magnetically responsive element is associated with the distal end of the elongate body. The mechanical properties of the elongate body 22 and the size and shape and position of the at least one magnetically responsive element are such that the magnetically navigable catheter can change shape in response to an application of a magnetic field or gradient. For example the magnetically navigable catheter can be configured so that the distal end of the catheter will substantially align with the direction of an applied magnetic field of 0.18 T or less.

At least one fiber optic sensor 30, and in this first embodiment, only one such sensor, extends substantially along the length of the elongate body 22 for use determining the location of at least one point along the length of the elongate body. This fiber optic sensor 30 may use low reflectance Fiber Bragg Grating (FBG) strain sensors to determine how any point along that fiber is positioned in space. The characteristics of optical fibers and the FBGs vary with curvature, and by sensing the relative change of FBGs in each of one or more fiber cores, the three-dimensional change in position can be determined. Examples of such sensors are disclosed in U.S. Pat. App. Publ. No. 2006/0013523 A1 (filed 13 Jul. 2005), U.S. Pat. App. Publ. No. 2007/0156019 A1 (filed 20 Jul. 2006), U.S. Pat. No. 8116601, the entire disclosures of which are incorporated by reference. Of course a fiber optic position or shape sensor employing some other mode of operation could be used in addition to, or instead of, such fiber optic sensor.

The at least one fiber optic sensor 30 preferably extends longitudinally along the elongate body 22, parallel to its longitudinal axis. Alternatively the fiber optic sensor 30 could be arranged differently with respect to the elongate body 22.

The fiber optic sensor 30 is preferably secured to the elongate body 22 by being embedded in a cladding 32, surrounding the elongate body 22. The cladding 32 is preferably a low friction, polymeric coating on at least the distal portions of the elongate body 22. The cladding 32 can have windows or openings therein for exposing sensors, such as sensing electrodes, or other structures, such as therapeutic electrodes, carried on the magnetically navigable catheter.

A second preferred embodiment of a magnetically navigable catheter with fiber optic position or shape sensor in accordance with the principles of this invention, indicated generally as 20B, is shown in transverse cross section in FIG. 2. The magnetically navigable catheter 20B comprises an elongate body 22 having a proximal end, and a distal end. At least one magnetically responsive element is associated with the distal end of the elongate body. The mechanical properties of the elongate body 22 and the size and shape and position of the at least one magnetically responsive element are such that the magnetically navigable catheter can change shape in response to an application of a magnetic field or gradient. For example the magnetically navigable catheter can be configured so that the distal end of the catheter will substantially align with the direction of an applied magnetic field of 0.18 T or less.

At least one fiber optic sensor 30, and in this second preferred embodiment two fiber optic sensors 30A and 30B extend substantially along the length of the elongate body 22 for use determining the location of at least one point along the length of the elongate body. These two fiber optic sensors are shown spaced at least 90° apart around the circumference of the elongate body 22, but they could be arranged at some other spacing, for example 180° apart. The fiber optic sensors 30 may be as described above with respect to the first preferred embodiment. The fiber optic sensors 30 preferably extend longitudinally along the elongate body 22, parallel to its longitudinal axis. Alternatively the fiber optic sensors 30 could be arranged differently with respect to the elongate body 22.

The fiber optic sensors 30 are preferably secured to the elongate body 22 by being embedded in a cladding 32, surrounding the elongate body 22. The cladding 32 is preferably a low friction, polymeric coating on at least the distal portions of the elongate body 22. The cladding 32 can have windows or openings therein for exposing sensors, such as sensing electrodes, or other structures, such as therapeutic electrodes, carried on the magnetically navigable catheter.

A third preferred embodiment of a magnetically navigable catheter with fiber optic position or shape sensor in accordance with the principles of this invention, indicated generally as 20C, is shown in transverse cross section in FIG. 3. The magnetically navigable catheter 20C comprises an elongate body 22 having a proximal end, and a distal end. At least one magnetically responsive element is associated with the distal end of the elongate body. The mechanical properties of the elongate body 22 and the size and shape and position of the at least one magnetically responsive element are such that the magnetically navigable catheter can change shape in response to an application of a magnetic field or gradient. For example the magnetically navigable catheter can be configured so that the distal end of the catheter will substantially align with the direction of an applied magnetic field of 0.18 T or less.

At least one fiber optic sensor 30, and in this third preferred embodiment three fiber optic sensors 30A, 30B, and 3C extend substantially along the length of the elongate body 22 for use determining the location of at least one point along the length of the elongate body. These three fiber optic sensors are shown spaced at least 90° apart around the circumference of the elongate body 22, but they could be arranged at some other spacing. The fiber optic sensors 30 may be as described above with respect to the first preferred embodiment. The fiber optic sensors 30 preferably extend longitudinally along the elongate body 22, parallel to its longitudinal axis. Alternatively the fiber optic sensors 30 could be arranged differently with respect to the elongate body 22, for example extending spirally around the elongate body.

The fiber optic sensors 30 are preferably secured to the elongate body 22 by being embedded in a cladding 32, surrounding the elongate body 22. The cladding 32 is preferably a low friction, polymeric coating on at least the distal portions of the elongate body 22. The cladding 32 can have windows or openings therein for exposing sensors, such as sensing electrodes, or other structures, such as therapeutic electrodes, carried on the magnetically navigable catheter.

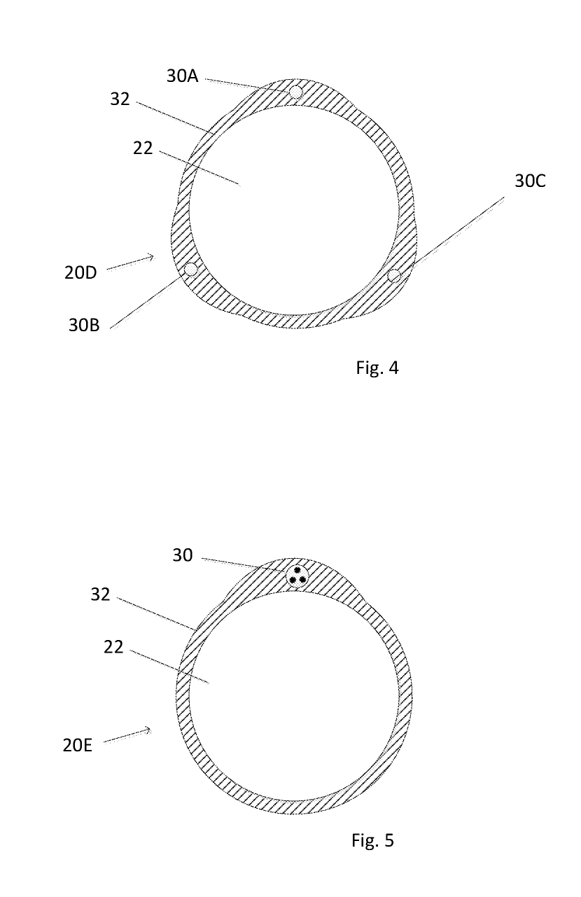

A fourth preferred embodiment of a magnetically navigable catheter with fiber optic position or shape sensor in accordance with the principles of this invention, indicated generally as 20D, is shown in transverse cross section in FIG. 4. Catheter 20D is similar to catheter 20C, except that rather than being spaced at 90° from each other, the fiber optic sensors 30 are equally spaced around the circumference of the elongate body 22 (i.e., at 120°).

A fifth preferred embodiment of a magnetically navigable catheter with fiber optic position or shape sensor in accordance with the principles of this invention, indicated generally as 20E, is shown in transverse cross section in FIG. 5. Catheter 20E is similar to catheter 20A, except that rather than a single fiber optic sensor 30, catheter 20E has three fiber optic sensors all ganged together at the same location.

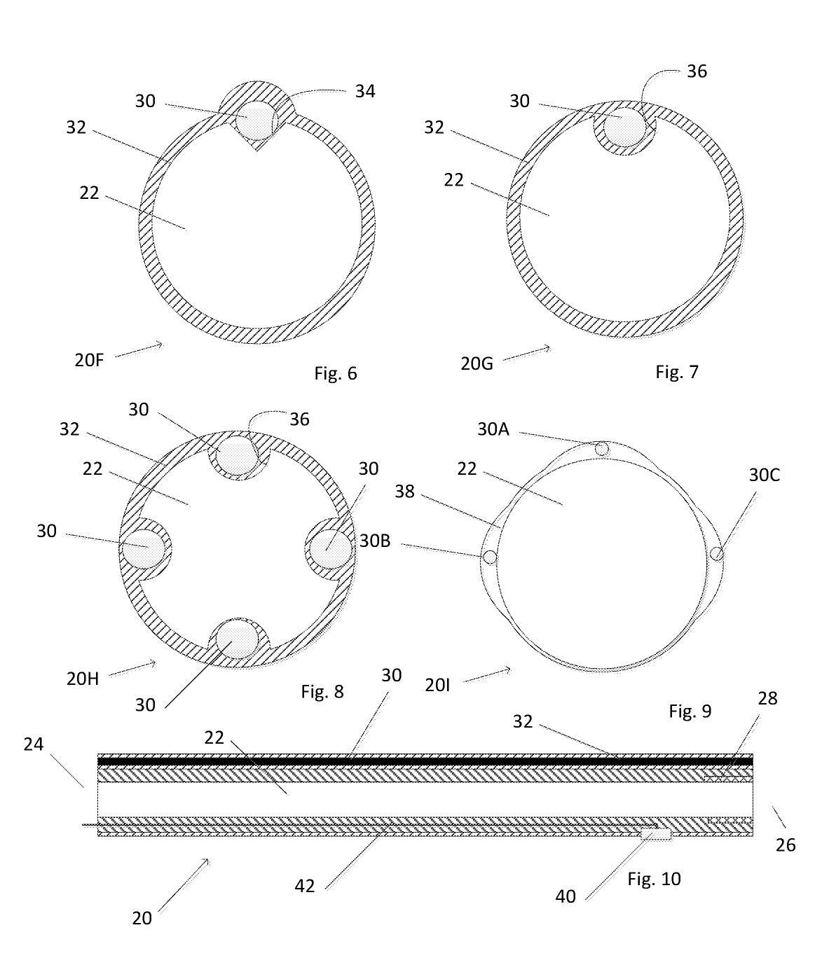

A sixth preferred embodiment of a magnetically navigable catheter with fiber optic position or shape sensor in accordance with the principles of this invention, indicated generally as 20F, is shown in transverse cross section in FIG. 6. The magnetically navigable catheter 20F comprises an elongate body 22 having a proximal end, and a distal end. At least one magnetically responsive element is associated with the distal end of the elongate body. The mechanical properties of the elongate body 22 and the size and shape and position of the at least one magnetically responsive element are such that the magnetically navigable catheter can change shape in response to an application of a magnetic field or gradient. For example the magnetically navigable catheter can be configured so that the distal end of the catheter will substantially align with the direction of an applied magnetic field of 0.18 T or less.

At least one fiber optic sensor 30, and in this sixth preferred embodiment just one fiber optic sensor 30 extends substantially along the length of the elongate body 22 for use determining the location of at least one point along the length of the elongate body. The elongate body may have at least one groove 34 formed therein for receiving at least a portion of the fiber optic sensor 30. As shown in FIG. 6, there is one v-shaped groove 34. The fiber optic sensor 30 may be as described above with respect to the first preferred embodiment. The fiber optic sensors 30 preferably extend longitudinally along the elongate body 22, parallel to its longitudinal axis. Alternatively the fiber optic sensor 30 could be arranged differently with respect to the elongate body 22, for example extending spirally around the elongate body, in which case the groove 34 would have a corresponding configuration.

The fiber optic sensors 30 are preferably secured to the elongate body 22 by being embedded in a cladding 32, surrounding the elongate body 22. The cladding 32 is preferably a low friction, polymeric coating on at least the distal portions of the elongate body 22. The cladding 32 can have windows or openings therein for exposing sensors, such as sensing electrodes, or other structures, such as therapeutic electrodes, carried on the magnetically navigable catheter.

A seventh preferred embodiment of a magnetically navigable catheter with fiber optic position or shape sensor in accordance with the principles of this invention, indicated generally as 20G, is shown in transverse cross section in FIG. 7. The magnetically navigable catheter 20G comprises an elongate body 22 having a proximal end, and a distal end. At least one magnetically responsive element is associated with the distal end of the elongate body. The mechanical properties of the elongate body 22 and the size and shape and position of the at least one magnetically responsive element are such that the magnetically navigable catheter can change shape in response to an application of a magnetic field or gradient. For example the magnetically navigable catheter can be configured so that the distal end of the catheter will substantially align with the direction of an applied magnetic field of 0.18 T or less.

At least one fiber optic sensor 30, and in this seventh preferred embodiment just one fiber optic sensor 30 extends substantially along the length of the elongate body 22 for use determining the location of at least one point along the length of the elongate body. The elongate body may have at least one groove 36 formed therein for receiving at least a portion of the fiber optic sensor 30. As shown in FIG. 6, there is one semicircular shaped groove 36. The fiber optic sensor 30 may be as described above with respect to the first preferred embodiment. The fiber optic sensors 30 preferably extend longitudinally along the elongate body 22, parallel to its longitudinal axis. Alternatively the fiber optic sensor 30 could be arranged differently with respect to the elongate body 22, for example extending spirally around the elongate body, in which case the groove 36 would have a corresponding configuration.

The fiber optic sensors 30 are preferably secured to the elongate body 22 by being embedded in a cladding 32, surrounding the elongate body 22. The cladding 32 is preferably a low friction, polymeric coating on at least the distal portions of the elongate body 22. The cladding 32 can have windows or openings therein for exposing sensors, such as sensing electrodes, or other structures, such as therapeutic electrodes, carried on the magnetically navigable catheter.

An eighth preferred embodiment of a magnetically navigable catheter with fiber optic position or shape sensor in accordance with the principles of this invention, indicated generally as 20H, is shown in transverse cross section in FIG. 8. The magnetically navigable catheter 20H is similar to catheter 20G, except that instead of one fiber optic sensor 30 and one groove 36, there are four fiber optic sensors, and four corresponding grooves, equally spaced around the circumference of the elongate body 22.

A ninth preferred embodiment of a magnetically navigable catheter with fiber optic position or shape sensor in accordance with the principles of this invention, indicated generally as 20I, is shown in transverse cross section in FIG. 9. The magnetically navigable catheter 20I comprises an elongate body 22 having a proximal end, and a distal end. At least one magnetically responsive element is associated with the distal end of the elongate body. The mechanical properties of the elongate body 22 and the size and shape and position of the at least one magnetically responsive element are such that the magnetically navigable catheter can change shape in response to an application of a magnetic field or gradient. For example the magnetically navigable catheter can be configured so that the distal end of the catheter will substantially align with the direction of an applied magnetic field of 0.18 T or less.

At least one fiber optic sensor 30, and in this third preferred embodiment three fiber optic sensors 30A, 30B, and 3C extend substantially along the length of the elongate body 22 for use determining the location of at least one point along the length of the elongate body. These three fiber optic sensors are shown spaced at least 90° apart around the circumference of the elongate body 22, but they could be arranged at some other spacing. The fiber optic sensors 30 may be as described above with respect to the first preferred embodiment. The fiber optic sensors 30 preferably extend longitudinally along the elongate body 22, parallel to its longitudinal axis. Alternatively the fiber optic sensors 30 could be arranged differently with respect to the elongate body 22, for example extending spirally around the elongate body.

The fiber optic sensors 30 are preferably secured to the elongate body 22 with a sleeve 38 enveloping the fiber optic sensors and the elongate body 22. The sleeve 38 can be a film or tube of a polymeric material, or it could be a mesh of a metallic or polymeric material, or some composite thereof. The sleeve 38 can have windows or openings therein for exposing sensors, such as sensing electrodes, or other structures, such as therapeutic electrodes, carried on the magnetically navigable catheter.

A tenth preferred embodiment of a magnetically navigable catheter with fiber optic position or shape sensor in accordance with the principles of this invention, indicated generally as 20, is shown in longitudinal cross section in FIG. 10, although this longitudinal cross section corresponds to catheter 20A, this description of the basic components is generally applicable to all embodiments. The magnetically navigable catheter 20 comprises an elongate body 22 having a proximal end 24, and a distal end 26. At least one magnetically responsive element 28 is associated with the distal end of the elongate body. This magnetically responsive element can be one or more discrete magnets incorporated at the distal end, or along the distal end portion of the catheter. Alternatively the magnetically responsive elements can be magnetically responsive material incorporated into portions of the catheter 20. Still another alternative for the magnetically responsive elements are small electromagnets incorporated at the distal end, or along the distal end portion of the catheter. As shown in FIG. 10, however, the magnetically responsive element 28 is a ring made of a magnetically responsive material embedded in the distal end of the elongate body 22.

The mechanical properties of the elongate body 22 and the size and shape and position of the at least one magnetically responsive element 28 are such that the magnetically navigable catheter can change shape in response to an application of a magnetic field or gradient. For example the magnetically navigable catheter can be configured so that the distal end of the catheter will substantially align with the direction of an applied magnetic field of 0.18 T or less.

At least one fiber optic sensor 30 extends substantially along the length of the elongate body 22 for use determining the location of at least one point along the length of the elongate body. The fiber optic sensors 30 may be as described above with respect to the first preferred embodiment. The fiber optic sensors 30 preferably extend longitudinally along the elongate body 22, parallel to its longitudinal axis. Alternatively the fiber optic sensors 30 could be arranged differently with respect to the elongate body 22, for example extending spirally around the elongate body.

The fiber optic sensor 30 is preferably secured to the elongate body 22 by being embedded in a cladding 32, surrounding the elongate body 22. The cladding 32 is preferably a low friction, polymeric coating on at least the distal portions of the elongate body 22. The cladding 32 can have windows or openings therein for exposing sensors, such as sensing electrodes, or other structures, such as therapeutic electrodes, carried on the magnetically navigable catheter. Alternatively, as described with respect to catheter 201, the fiber optic sensors 30 can be secured to the elongate body 22 with a sleeve 38 enveloping the fiber optic sensors and the elongate body 22. The sleeve 38 can be a film or tube of a polymeric material, or it could be a mesh of a metallic or polymeric material, or some composite thereof. The sleeve 38 can have windows or openings therein for exposing sensors, such as sensing electrodes, or other structures, such as therapeutic electrodes, carried on the magnetically navigable catheter.

An electrode 40 is preferably provided on the exterior of the elongate body, to act as a sensor or to delivery therapy such as electrophysiological pacing or tissue ablation. The electrode is connected by wires 42 extending to the proximal end of the catheter 20 to connect to appropriate equipment for sensing or application of therapy. Similarly, the proximal end of the at least one fiber optic sensor is connected to appropriate equipment, for example a laser and sensor, for determining the position or shape of the fiber optic element, and thus the magnetic navigation catheter with which it is associated.

Operation

In operation, the magnetic catheter can be navigated as it normally would with the aid of a magnetic navigation system. However, the fiber optic position sensors can provide position information, for example for the distal end of the catheter, or of the various electrodes 40. This position can be used as feed back in navigating to preselected target locations in the body.

Alternatively the fiber optic position sensors can be used in mapping, determining the position of the distal end or distal end portion of the magnetically navigated catheter, and thereby determining the shape of an anatomical structure with which the catheter is in contact, and even associating location information with physiologic information, for example sensed electrophysiology information.

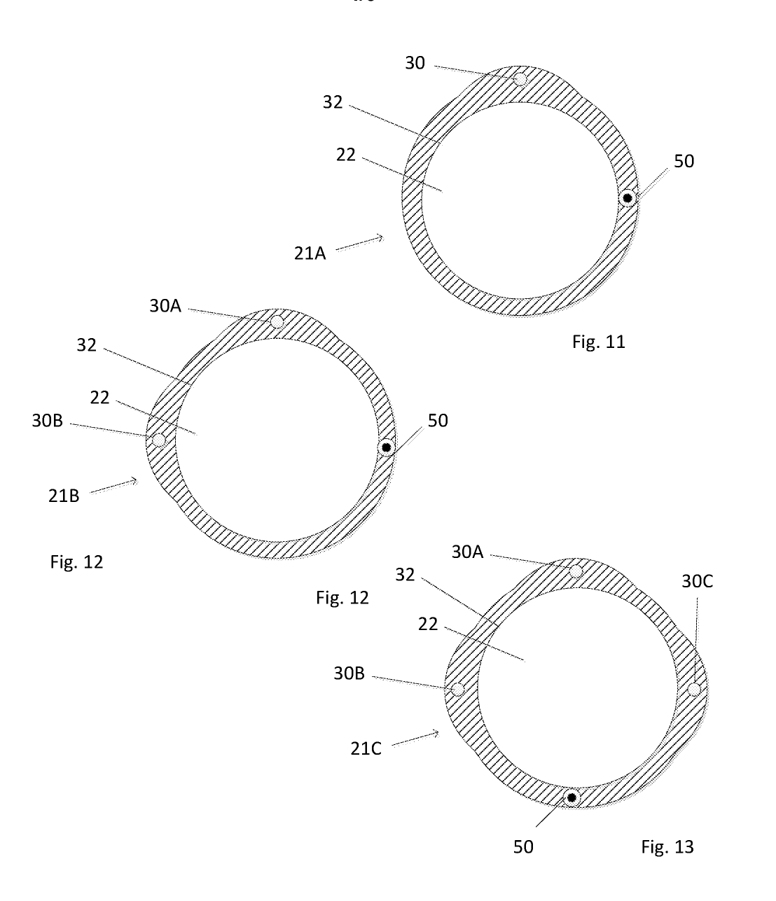

A first preferred embodiment of a mechanically navigable catheter with fiber optic position or shape sensor in accordance with the principles of this invention, indicated generally as 21A, is shown in transverse cross section in FIG. 11. The mechanically navigable catheter 21A is similar in construction to catheter 20A, and corresponding parts are identified by corresponding reference numerals. The mechanically navigable catheter 21A comprises an elongate body 22 having a proximal end, and a distal end. At least one mechanically responsive element (not shown) is associated with the distal end of the elongate body. The mechanical properties of the elongate body 22 and the size and shape and position of the at least one mechanically responsive element are such that the mechanically navigable catheter can change shape in response to operation of one or more pull wires or push wires 50 slidably disposed in a passage in the wall of the elongate body.

At least one fiber optic sensor 30, and in this first embodiment, only one such sensor, extends substantially along the length of the elongate body 22 for use determining the location of at least one point along the length of the elongate body. This fiber optic sensor 30 may use low reflectance Fiber Bragg Grating (FBG) strain sensors to determine how any point along that fiber is positioned in space. The characteristics of optical fibers and the FBGs vary with curvature, and by sensing the relative change of FBGs in each of one or more fiber cores, the three-dimensional change in position can be determined. Examples of such sensors are disclosed in U.S. Pat App. Publ. No. 2006/0013523 A1 (filed 13 Jul. 2005), U.S. Pat. App. Publ. No. 2007/0156019 A1 (filed 20 Jul. 2006), U.S. Pat. No. 8116601, the entire disclosures of which are incorporated by reference. Of course a fiber optic position or shape sensor employing some other mode of operation could be used in addition to, or instead of, such fiber optic sensor.

The at least one fiber optic sensor 30 preferably extends longitudinally along the elongate body 22, parallel to its longitudinal axis. Alternatively the fiber optic sensor 30 could be arranged differently with respect to the elongate body 22.

The fiber optic sensor 30 is preferably secured to the elongate body 22 by being embedded in a cladding 32, surrounding the elongate body 22. The cladding 32 is preferably a low friction, polymeric coating on at least the distal portions of the elongate body 22. The cladding 32 can have windows or openings therein for exposing sensors, such as sensing electrodes, or other structures, such as therapeutic electrodes, carried on the magnetically navigable catheter.

A second preferred embodiment of a magnetically navigable catheter with fiber optic position or shape sensor in accordance with the principles of this invention, indicated generally as 21B, is shown in transverse cross section in FIG. 12. The mechanically navigable catheter 21B comprises an elongate body 22 having a proximal end, and a distal end. At least one mechanically responsive element is associated with the distal end of the elongate body. The mechanical properties of the elongate body 22 and the size and shape and position of the at least one mechanically responsive element are such that the mechanically navigable catheter can change shape in response to operation of one or more pull wires or push wires 50 slidably disposed in a passage in the wall of the elongate body.

At least one fiber optic sensor 30, and in this second preferred embodiment two fiber optic sensors 30A and 30B extend substantially along the length of the elongate body 22 for use determining the location of at least one point along the length of the elongate body. These two fiber optic sensors are shown spaced at least 90° apart around the circumference of the elongate body 22, but they could be arranged at some other spacing, for example 180° apart. The fiber optic sensors 30 may be as described above with respect to the first preferred embodiment. The fiber optic sensors 30 preferably extend longitudinally along the elongate body 22, parallel to its longitudinal axis. Alternatively the fiber optic sensors 30 could be arranged differently with respect to the elongate body 22.

The fiber optic sensors 30 are preferably secured to the elongate body 22 by being embedded in a cladding 32, surrounding the elongate body 22. The cladding 32 is preferably a low friction, polymeric coating on at least the distal portions of the elongate body 22. The cladding 32 can have windows or openings therein for exposing sensors, such as sensing electrodes, or other structures, such as therapeutic electrodes, carried on the mechanically navigable catheter.

A third preferred embodiment of a mechanically navigable catheter with fiber optic position or shape sensor in accordance with the principles of this invention, indicated generally as 21C, is shown in transverse cross section in FIG. 13. The mechanically navigable catheter 21C comprises an elongate body 22 having a proximal end, and a distal end. At least one mechanically responsive element is associated with the distal end of the elongate body. The mechanical properties of the elongate body 22 and the size and shape and position of the at least one mechanically responsive element are such that the mechanically navigable catheter can change shape in response to operation of one or more pull wires or push wires 50 slidably disposed in a passage in the wall of the elongate body.

At least one fiber optic sensor 30, and in this third preferred embodiment three fiber optic sensors 30A, 30B, and 3C extend substantially along the length of the elongate body 22 for use determining the location of at least one point along the length of the elongate body. These three fiber optic sensors are shown spaced at least 90° apart around the circumference of the elongate body 22, but they could be arranged at some other spacing. The fiber optic sensors 30 may be as described above with respect to the first preferred embodiment. The fiber optic sensors 30 preferably extend longitudinally along the elongate body 22, parallel to its longitudinal axis. Alternatively the fiber optic sensors 30 could be arranged differently with respect to the elongate body 22, for example extending spirally around the elongate body.

The fiber optic sensors 30 are preferably secured to the elongate body 22 by being embedded in a cladding 32, surrounding the elongate body 22. The cladding 32 is preferably a low friction, polymeric coating on at least the distal portions of the elongate body 22. The cladding 32 can have windows or openings therein for exposing sensors, such as sensing electrodes, or other structures, such as therapeutic electrodes, carried on the mechanically navigable catheter.

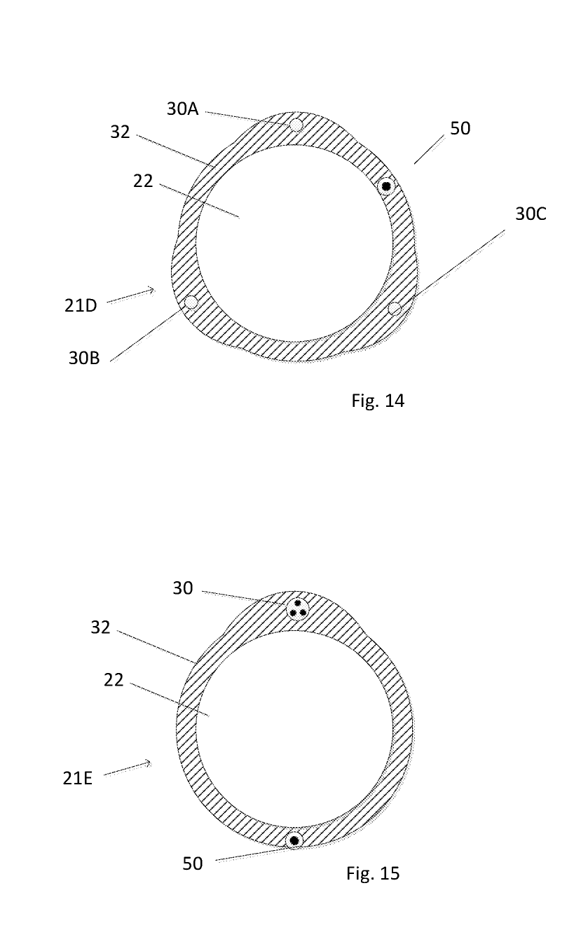

A fourth preferred embodiment of a mechanically navigable catheter with fiber optic position or shape sensor in accordance with the principles of this invention, indicated generally as 21C, is shown in transverse cross section in FIG. 14. Catheter 21D is similar to catheter 21C, except that rather than being spaced at 90° from each other, the fiber optic sensors 30 are equally spaced around the circumference of the elongate body 22 (i.e., at 120°).

A fifth preferred embodiment of a mechanically navigable catheter with fiber optic position or shape sensor in accordance with the principles of this invention, indicated generally as 21E, is shown in transverse cross section in FIG. 15. Catheter 21E is similar to catheter 21A, except that rather than a single fiber optic sensor 30, catheter 21E has three fiber optic sensors all ganged together at the same location.

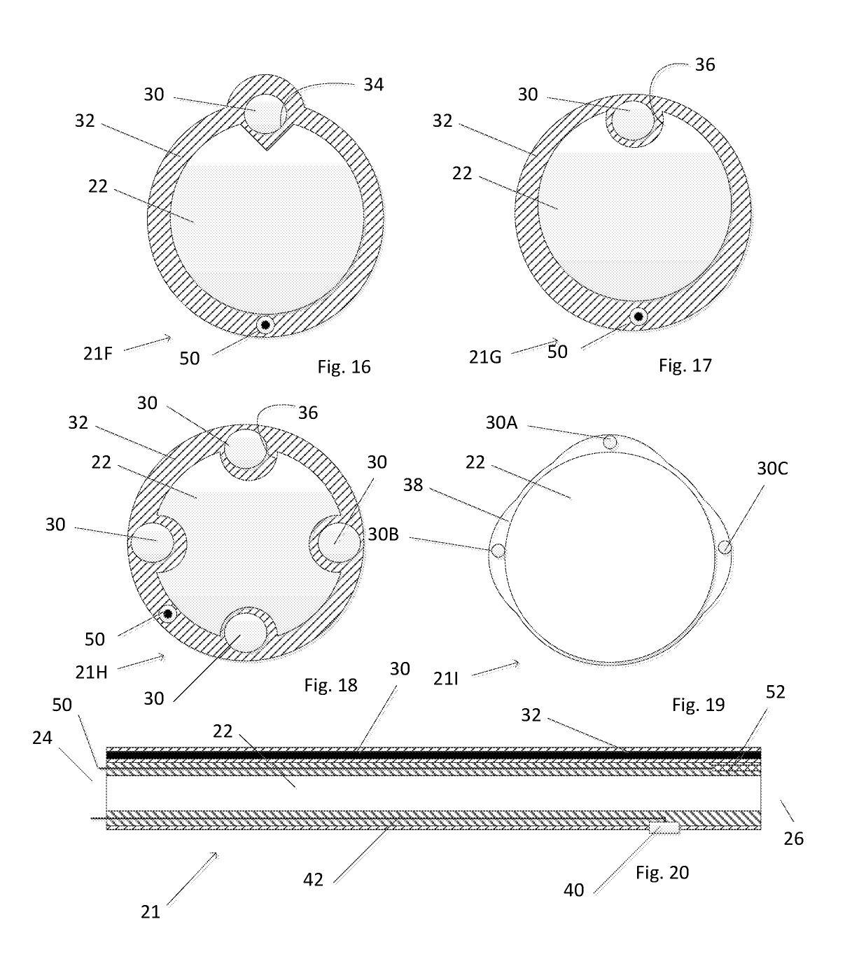

A sixth preferred embodiment of a mechanically navigable catheter with fiber optic position or shape sensor in accordance with the principles of this invention, indicated generally as 21F, is shown in transverse cross section in FIG. 16. The mechanically navigable catheter 21F comprises an elongate body 22 having a proximal end, and a distal end. At least one mechanically responsive element is associated with the distal end of the elongate body. The mechanical properties of the elongate body 22 and the size and shape and position of the at least one mechanically responsive element are such that the mechanically navigable catheter can change shape in response to operation of one or more pull wires or push wires 50 slidably disposed in a passage in the wall of the elongate body.

At least one fiber optic sensor 30, and in this sixth preferred embodiment just one fiber optic sensor 30 extends substantially along the length of the elongate body 22 for use determining the location of at least one point along the length of the elongate body. The elongate body may have at least one groove 34 formed therein for receiving at least a portion of the fiber optic sensor 30. As shown in FIG. 16, there is one v-shaped groove 34. The fiber optic sensor 30 may be as described above with respect to the first preferred embodiment. The fiber optic sensors 30 preferably extend longitudinally along the elongate body 22, parallel to its longitudinal axis. Alternatively the fiber optic sensor 30 could be arranged differently with respect to the elongate body 22, for example extending spirally around the elongate body, in which case the groove 34 would have a corresponding configuration.

The fiber optic sensors 30 are preferably secured to the elongate body 22 by being embedded in a cladding 32, surrounding the elongate body 22. The cladding 32 is preferably a low friction, polymeric coating on at least the distal portions of the elongate body 22. The cladding 32 can have windows or openings therein for exposing sensors, such as sensing electrodes, or other structures, such as therapeutic electrodes, carried on the mechanically navigable catheter.

A seventh preferred embodiment of a mechanically navigable catheter with fiber optic position or shape sensor in accordance with the principles of this invention, indicated generally as 20G, is shown in transverse cross section in FIG. 17. The mechanically navigable catheter 20G comprises an elongate body 22 having a proximal end, and a distal end. At least one mechanically responsive element is associated with the distal end of the elongate body. The mechanical properties of the elongate body 22 and the size and shape and position of the at least one mechanically responsive element are such that the mechanically navigable catheter can change shape in response to operation of one or more pull wires or push wires 50 slidably disposed in a passage in the wall of the elongate body.

At least one fiber optic sensor 30, and in this seventh preferred embodiment just one fiber optic sensor 30 extends substantially along the length of the elongate body 22 for use determining the location of at least one point along the length of the elongate body. The elongate body may have at least one groove 36 formed therein for receiving at least a portion of the fiber optic sensor 30. As shown in FIG. 16, there is one semicircular shaped groove 36. The fiber optic sensor 30 may be as described above with respect to the first preferred embodiment. The fiber optic sensors 30 preferably extend longitudinally along the elongate body 22, parallel to its longitudinal axis. Alternatively the fiber optic sensor 30 could be arranged differently with respect to the elongate body 22, for example extending spirally around the elongate body, in which case the groove 36 would have a corresponding configuration.

The fiber optic sensors 30 are preferably secured to the elongate body 22 by being embedded in a cladding 32, surrounding the elongate body 22. The cladding 32 is preferably a low friction, polymeric coating on at least the distal portions of the elongate body 22. The cladding 32 can have windows or openings therein for exposing sensors, such as sensing electrodes, or other structures, such as therapeutic electrodes, carried on the mechanically navigable catheter.

An eighth preferred embodiment of a mechanically navigable catheter with fiber optic position or shape sensor in accordance with the principles of this invention, indicated generally as 21H, is shown in transverse cross section in FIG. 18. The mechanically navigable catheter 21H is similar to catheter 21G, except that instead of one fiber optic sensor 30 and one groove 36, there are four fiber optic sensors, and four corresponding grooves, equally spaced around the circumference of the elongate body 22.

A ninth preferred embodiment of a mechanically navigable catheter with fiber optic position or shape sensor in accordance with the principles of this invention, indicated generally as 21I, is shown in transverse cross section in FIG. 9. The mechanically navigable catheter 21I comprises an elongate body 22 having a proximal end, and a distal end. At least one mechanically responsive element is associated with the distal end of the elongate body.

At least one fiber optic sensor 30, and in this third preferred embodiment three fiber optic sensors 30A, 30B, and 3C extend substantially along the length of the elongate body 22 for use determining the location of at least one point along the length of the elongate body. These three fiber optic sensors are shown spaced at least 90° apart around the circumference of the elongate body 22, but they could be arranged at some other spacing. The fiber optic sensors 30 may be as described above with respect to the first preferred embodiment. The fiber optic sensors 30 preferably extend longitudinally along the elongate body 22, parallel to its longitudinal axis. Alternatively the fiber optic sensors 30 could be arranged differently with respect to the elongate body 22, for example extending spirally around the elongate body.

The fiber optic sensors 30 are preferably secured to the elongate body 22 with a sleeve 38 enveloping the fiber optic sensors and the elongate body 22. The sleeve 38 can be a film or tube of a polymeric material, or it could be a mesh of a metallic or polymeric material, or some composite thereof. The sleeve 38 can have windows or openings therein for exposing sensors, such as sensing electrodes, or other structures, such as therapeutic electrodes, carried on the mechanically navigable catheter.

A tenth preferred embodiment of a mechanically navigable catheter with fiber optic position or shape sensor in accordance with the principles of this invention, indicated generally as 21, is shown in longitudinal cross section in FIG. 20, although this longitudinal cross section corresponds to catheter 21A, this description of the basic components is generally applicable to all embodiments. The mechanically navigable catheter 21 comprises an elongate body 22 having a proximal end 24, and a distal end 26. At least one mechanically responsive element 52 is associated with the distal end of the elongate body. The mechanical properties of the elongate body 22 and the size and shape and position of the at least one mechanically responsive element are such that the mechanically navigable catheter can change shape in response to operation of one or more pull wires or push wires 50 slidably disposed in a passage in the wall of the elongate body.

At least one fiber optic sensor 30 extends substantially along the length of the elongate body 22 for use determining the location of at least one point along the length of the elongate body. The fiber optic sensors 30 may be as described above with respect to the first preferred embodiment. The fiber optic sensors 30 preferably extend longitudinally along the elongate body 22, parallel to its longitudinal axis. Alternatively the fiber optic sensors 30 could be arranged differently with respect to the elongate body 22, for example extending spirally around the elongate body.

The fiber optic sensor 30 is preferably secured to the elongate body 22 by being embedded in a cladding 32, surrounding the elongate body 22. The cladding 32 is preferably a low friction, polymeric coating on at least the distal portions of the elongate body 22. The cladding 32 can have windows or openings therein for exposing sensors, such as sensing electrodes, or other structures, such as therapeutic electrodes, carried on the mechanically navigable catheter. Alternatively, as described with respect to catheter 21I, the fiber optic sensors 30 can be secured to the elongate body 22 with a sleeve 38 enveloping the fiber optic sensors and the elongate body 22. The sleeve 38 can be a film or tube of a polymeric material, or it could be a mesh of a metallic or polymeric material, or some composite thereof. The sleeve 38 can have windows or openings therein for exposing sensors, such as sensing electrodes, or other structures, such as therapeutic electrodes, carried on the mechanically navigable catheter.

An electrode 40 is preferably provided on the exterior of the elongate body, to act as a sensor or to delivery therapy such as electrophysiological pacing or tissue ablation. The electrode is connected by wires 42 extending to the proximal end of the catheter 21 to connect to appropriate equipment for sensing or application of therapy. Similarly, the proximal end of the at least one fiber optic sensor is connected to appropriate equipment, for example a laser and sensor, for determining the position or shape of the fiber optic element, and thus the mechanical navigation catheter with which it is associated.

Of course, a catheter can be made to be both mechanically and magnetically navigable, to facilitate the navigation and control of the catheter, with the position and configuration feedback used to automate navigation in a subject.

Operation

In operation, the mechanical catheter can be navigated as it normally would, either by manual operation of controls that operate the pull wires or push wires 50, or through an interface the controls that operate the pull wires or push wires. The fiber optic position sensors can provide position information, for example for the distal end of the catheter, or of the various electrodes 40. This position can be used as feed back in manually or automatically navigating to preselected target locations in the body.

Alternatively the fiber optic position sensors can be used in mapping, determining the position of the distal end or distal end portion of the mechanically navigated catheter, and thereby determining the shape of an anatomical structure with which the catheter is in contact, and even associating location information with physiologic information, for example sensed electrophysiology information.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A magnetically navigable catheter comprising:
an elongate body having a proximal and a distal end;
at least one magnetically responsive element associated with the distal end of the elongate body; and
at least one fiber optic sensor extending substantially along the length of the elongate body for use determining the location of at least one point along the length of the elongate body.

2. The magnetically navigable catheter according to claim 1 wherein there are at least three fiber optic sensor extending substantially along the length of the elongate body.

3. The magnetically navigable catheter according to claim 2 wherein the fiber optic sensor extend longitudinally, parallel to the longitudinal axis of the elongate body.

4. The magnetically navigable catheter according to claim 2 wherein the fiber optic sensors extend spirally around the longitudinal axis of the elongate body.

5. The magnetically navigable catheter according to claim 2 wherein the fiber optic sensors are spaced equally around the cross-sectional perimeter of the elongate body.

6. The magnetically navigable catheter according to claim 2 wherein there are at least first, second and third fiber optic sensors, the second and third sensors being offset 90° on opposite sides of the first sensor.

7. The magnetically navigable catheter according to claim 2 further comprising a cladding surrounding the elongate body, and wherein the fiber optic sensors are embedded into the cladding.

8. The magnetically navigable catheter according to claim 2 wherein at least two of the fiber optic sensors are adjacent to each other.

9. The magnetically navigable catheter according to claim 8 wherein all of the fiber optic sensors are adjacent to each other.

10. The magnetically navigable catheter according to claim 2 wherein the fiber optic sensors are secured on the outside of the elongate body with a sleeve enveloping the fiber optic sensors and the elongate body.

11. The magnetically navigable catheter according to claim 10 wherein the sleeve comprises a polymeric web material.

12. The magnetically navigable catheter according to claim 10 wherein the sleeve is a mesh material.

13. The magnetically navigable catheter according to claim 12 wherein the mesh is a metallic mesh.

14. The magnetically navigable catheter according to claim 12 wherein the mesh is a polymeric mesh.

15. The magnetically navigable catheter according to claim 10 wherein the elongate body has at least one groove therein, at least partially receiving at least one fiber optic sensor.

16. The magnetically navigable catheter according to claim 15 wherein the elongate body has a plurality of grooves therein, each at least partially receiving at least one fiber optic sensor.

17. A mechanically navigable catheter comprising:
an elongate body having a proximal and a distal end;
at least one mechanically responsive element associated with the distal end of the elongate body for changing the configuration of the elongate body;
at least one elongate element extending along at least a portion of the length of the elongate body for operating the at least one mechanically responsive element; and
at least one fiber optic sensor extending substantially along the length of the elongate body for use determining the location of at least one point along the length of the elongate body.

18. The mechanically navigable catheter according to claim 17 wherein there are at least three fiber optic sensor extending substantially along the length of the elongate body.

19. The mechanically navigable catheter according to claim 18 wherein the fiber optic sensor extend longitudinally, parallel to the longitudinal axis of the elongate body.

20. The mechanically navigable catheter according to claim 18 wherein the fiber optic sensors extend spirally around the longitudinal axis of the elongate body.

21. The mechanically navigable catheter according to claim 18 wherein the fiber optic sensors are spaced equally around the cross-sectional perimeter of the elongate body.

22. The mechanically navigable catheter according to claim 18 wherein there are at least first, second and third fiber optic sensors, the second and third sensors being offset 90° on opposite sides of the first sensor.

23. The mechanically navigable catheter according to claim 18 further comprising a cladding surrounding the elongate body, and wherein the fiber optic sensors are embedded into the cladding.

24. The mechanically navigable catheter according to claim 18 wherein at least two of the fiber optic sensors are adjacent to each other.

25. The mechanically navigable catheter according to claim 24 wherein all of the fiber optic sensors are adjacent to each other.

26. The mechanically navigable catheter according to claim 18 wherein the fiber optic sensors are secured on the outside of the elongate body with a sleeve enveloping the fiber optic sensors and the elongate body.

27. The mechanically navigable catheter according to claim 26 wherein the sleeve comprises a polymeric web material.

28. The mechanically navigable catheter according to claim 26 wherein the sleeve is a mesh material.

29. The mechanically navigable catheter according to claim 28 wherein the mesh is a metallic mesh.

30. The mechanically navigable catheter according to claim 28 wherein the mesh is a polymeric mesh.

31. The mechanically navigable catheter according to claim 18 wherein the elongate body has at least one groove therein, at least partially receiving at least one fiber optic sensor.

32. The mechanically navigable catheter according to claim 31 wherein the elongate body has a plurality of grooves therein, each at least partially receiving at least one fiber optic sensor.
